(19) United States
(12) Patent Application Publication
(10) Pub. No.: US 2023/0330036 A1
(43) Pub. Date: Oct. 19, 2023

(54) LONG ACTING, CONTINUOUS ORAL RELEASE FROM ORAL DISPERSING STRIPS (ODS) ADDRESSING THE NEED FOR HIGH DOSAGE OF ACTIVE INGREDIENTS

(71) Applicant: Oak Therapeutics, Inc., Oxnard, CA (US)

(72) Inventors: Zachary Nicolay, Oxnard, CA (US); Edward Maliski, Oxnard, CA (US)

(21) Appl. No.: 18/300,281

(22) Filed: Apr. 13, 2023

Related U.S. Application Data
(60) Provisional application No. 63/330,668, filed on Apr. 13, 2022.

(51) Int. Cl. A61K 31/047; A61K 47/10; A61K 47/26; A61K 47/32; A61K 47/69; A61K 9/00

(52) U.S. Cl. CPC A61K 31/047; A61K 47/10; A61K 47/26; A61K 47/32; A61K 47/6939; A61K 9/0056

(57) ABSTRACT

There is disclosed a method of making a physical oral delivery system of an oral delivery strip for delivering an active ingredient over a long period of time.

### RELATED APPLICATION INFORMATION

This patent claims priority from the following provisional patent application No. 63/330,668 filed Apr. 13, 2022.

### NOTICE OF COPYRIGHTS AND TRADE DRESS

A portion of the disclosure of this patent document contains material which is subject to copyright protection. This patent document may show and/or describe matter which is or may become trade dress of the owner. The copyright and trade dress owner has no objection to the facsimile reproduction by anyone of the patent disclosure as it appears in the Patent and Trademark Office patent files or records, but otherwise reserves all copyright and trade dress rights whatsoever.

### BACKGROUND

### Field

This disclosure relates to oral dispersing strips. More specifically to methods and apparatuses for delivering medications to the oral cavity itself to a patient in need thereof over a long period of time and at a steady rate especially when sleeping or during other times of unconsciousness.

### Description of the Related Art

Other technology has tried to grapple with the issue of steady release of drugs or active ingredients but has failed for a multitude of reasons. There are several inventions that address the idea of continuous delivery of active(s) to the oral cavity. The solutions found in the literature include devices placed in the oral cavity. The devices are loaded with a slow dissolving formulation that releases the active(s) as they dissolve over time. These devices can be uncomfortable and awkward to use.

Methods such as dissolvable tablets require two tablets be adhered to a tooth for overnight use. Such a hard substance rubbing against the cheek is very uncomfortable to most patients. Furthermore, this method does not usually last for a complete sleep cycle (7-8 hours). This results in patients not taking their medication or not having medication administered effectively. Other forms of overnight treatments such as gels can be hard to keep in one place leading to a potential choking hazard or at least waking the patient from sleep. These other methods also fail to deliver consistent dosage delivery for a full 8 hours.

Furthermore, other inventions require xanthan gum. Xanthan gum is a polysaccharide used in many industrial applications, including food additives. Xanthan gum when mixed in with as little as 1% weight or volume of solution can increase the viscosity of a liquid. Other inventions that have tried to release chemicals at a constant rate have utilized xanthan gum but at a huge trade off. Modern research has linked xanthan gum to respiratory and digestive problems whereas some patients are allergic to the chemical. There is also research that indicates people with gluten allergies or intolerances have a hard time digesting xanthan gum.

Furthermore, xanthan gum has a high shear thinning or pseudoplasticity, meaning the viscosity of xanthan gum solutions tends to decrease with higher shear rates. So, when xanthan gum solutions are exposed to shear stress (such as from chewing or being inside a mouth) the viscosity and thus drug release of solutions utilizing xanthan gum changes. This makes it nearly impossible for xanthan gum based release systems to release chemicals at a steady state for a prolonged period of time. The longer the solution is in the mouth, the more the rate of drug release changes until it reaches a rate of zero release. This is not ideal for drug delivery systems that need to release a drug at a steady rate for over 8 hours.

Other inventions have used in situ hydrogel formations. Unfortunately, these solutions have never been able to release drugs for a long period of time (for example some only get to 5 mg of active ingredient for a period of 3 hours). This is likely due to the fact that hydrogel formations come in near infinite combinations. Hydrogels are degradable polymeric networks, in which cross-links at different areas of the polymers making up the network affect formation and degradation. Because of the thousands of different polymers and different linking areas, there are too many different combinations for them all to be tested. Furthermore, most if not all hydrogels are hydrous or at least contain some form of water. The present disclosure utilizes strips made from anhydrous hydrogels. Not only does this help with uniform drug release but this keeps the overall size of the strip smaller.

Other inventions have used in situ hydrogel formations. Unfortunately, these solutions have never been able to release drugs for a long period of time (for example some only get to 5 mg of active ingredient for a period of 3 hours). This is likely due to the fact that hydrogel formations come in near infinite combinations. Hydrogels are degradable polymeric networks, in which cross-links at different areas of the polymers making up the network affect formation and degradation. Because of the thousands of different polymers and different linking areas, there are too many different combinations for them all to be tested. Furthermore, most if not all hydrogels are hydrous or at least contain some form of water. The present disclosure utilizes strips made from anhydrous hydrogels. Not only does this help with uniform drug release but this keeps the overall size of the strip smaller.

### DESCRIPTION OF THE DRAWINGS

FIG. 1 is a picture of the chemical structure of Xylitol.

FIG. 2 is a picture of a graph showing the release of active ingredient with the present disclosure and other products.

FIG. 3 is a picture of a graph showing the first order decay of active released from a Xerostrip.

FIG. 4 is a diagram of the chemical structure of a molecule of vinylpyrrolidone with hydrogen atoms exposed.

FIG. 5 is a diagram of the chemical structure of sucralose.

### DETAILED DESCRIPTION

This disclosure relates to drug and chemical delivery systems. Specifically, the present application discloses compositions, methods of manufacture, products and methods of use relating to films containing either or both pharmaceutical and non-pharmaceutical therapeutic additives.

The disclosure more particularly relates the continuous and uniform oral delivery of the active(s) ingredient, chemical, drug, or other composition dispersed from an oral dispersing strip (ODS also referred to as oral delivery system or other abbreviations and structures as discussed below) adhered to tissue in the oral cavity. It should be noted that the ODS may also be applied to cavities or openings in the body that are not oral. Moreover, the present disclosure ODS maintains a stable delivery for as little as 5 minutes or for a period of 8 or more hours. The delivery of substances from the ODS is independent of the patient's state of consciousness. Further the ODS accommodates up to 500 mg or more of actives (otherwise known as active ingredient, chemical, drug, or other composition) yet is small enough to fit comfortably within the oral cavity even during sleep.

The present disclosure has a multitude of applications. One such application is treatment of dry mouth xerostomia. Xerostomia is a debilitating condition characterized by the subjective feeling of dry mouth. It is often associated with hyposalivation with prevalence ranging from 8% to 64.9% of the population. In patients that have Sjogren's disease or patients that have received head and neck radiation therapy, the prevalence of xerostomia nears 100%.

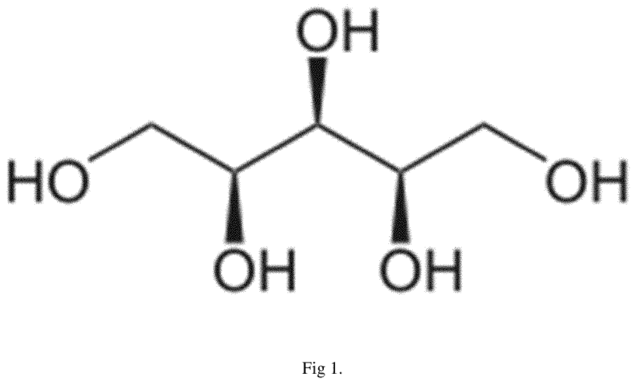

To treat this disease, this disclosure discloses a method of making XeroStrips (referred to as ODS, the present disclosure, and present ODS) that can be applied to the mouth. Xylitol is a naturally occurring sugar alcohol found in most plant material including many fruits and vegetables. It contains anti-cariogenic properties and acts to stimulate salivary flow by stimulating salivary gland secretions. A chemical formula for Xylitol may be seen in FIG. 1.

Current marketed products that contain Xylitol are limited in effective application. For one, none of them have actually been successful in treating dry mouth during sleep beyond 4-6 hours. This is because current products dissolve too quickly. XeroStrip and the current disclosure is able to provide xylitol continuously for more than 8 hours. This unique solution allows patients that suffer from severe dry mouth to have a good, uninterrupted night's sleep and provides patient's dentition with anticariogenic properties. Additionally, xylitol in high doses can cause diarrhea especially in cancer patients. The current disclosure's lower dosing mitigates that problem.

The current disclosure may be formed via the combination of an anhydrous binder, water activated gelling agent, solvent, and ancillary compound. Appropriate anhydrous binders include polyvinylpyrrolidone (Kollidon 90F), polyvinylpyrrolidone Mw 20-58K, and Hypromellose—100K. Additional anhydrous binders include: Hypromellose 2910, Hydroxypropyl Cellulose Mw 20-100K, Polyvinyl acetate-Polyvinylpyrrolidone mixture (Kollidon SR), polyvinyl caprolactam-polyvinyl acetate-polyethylene glycol graft co-polymer (Soluplus). Note in certain embodiments it may be beneficial to combine or change proportions or use only a single anhydrous binder. For example, a 30%-50% polyvinylpyrrolidone (Kollidon 90F) and 50% polyvinylpyrrolidone Mw 20-58K binder may be used.

Several water activated gelling agents may also be used. Appropriate water gelling agents include: Carboxymethyl cellulose—high Mw, vinylpyrrolidone-vinyl acetate (Kollidon VA 64), Methyl Oxirane polymer with Oxirane (Kolliphor-407). Additional water activated gelling agents include: Hyaluronic acid, Poloxamer Polyoxyethylene (160), Polyoxypropylene (30) Glycol) (Kolliphor 188)], Polyvinyl Alcohol Mw 10-30K, Sodium Alginate, Carbomer Polymers, Carrageenan Polymers, Pullulan, Pectin, polyvinylpyrrolidone, Gellan Gum, & Agar gum. Note different proportions of gelling agents may be used with each other. For example, 33% Carboxymethyl cellulose—high Mw, 33% vinylpyrrolidone-vinyl acetate (Kollidon VA 64), 33% Polyoxypropylene (30).

Multiple solvents may also be used. Absolute Ethanol, Ethanol, 200 proof ethanol, Glycerin, and PEG may be used. Additional solvents including Acetone, Triglycerides, Palm Oil, Mineral Oil, ethyl acetate, and other alcohols may also be used. In the experience of the inventors, it is beneficial to only have one solvent rather than several, but combinations may also work.

Experimental data confirms the efficacy of the present disclosure's ODS (oral delivery system). One experimental study treated 13 test subjects with the present disclosure. Qualitative data was collected from the patients including the patients' dry mouth level and improvement in sleep quality. The results conclusively show that application of the ODS improved patient outcome. Patients in the study were seeing relief from an ODS loaded with 300 mg Xylitol per strip overnight. When compared to the control that did not use the present ODS, patients that used two 550 mg tablets at night did not receive nearly the same benefit.

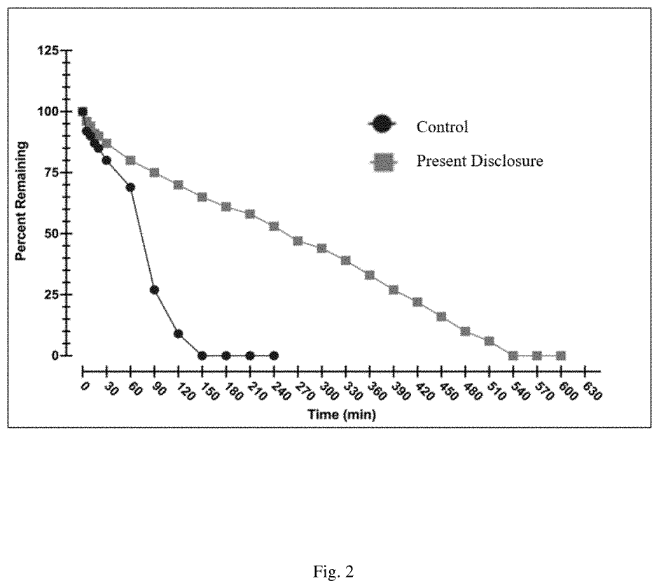

Another study conducted measured the oral release profile of the present ODS with a control made from a different formulation, XyliMelts® (Quest) oral dissolving tabs (the control). The different formulation was composed of acacia gum, cellulose gum, hydroxypropylcellulose, natural mint flavors, magnesium stearate, sodium bicarbonate, calcium carbonate. The study showed that the control oral dissolving tabs released its active ingredient in 2 hours (T₉₀). The present ODS continued to release its active ingredient for 8 hours (T₉₀). Mucosal adhesion during the over 8 hours of release was also maintained for the present ODS. The results compared may be seen in FIG. 2.

The following table contains the data found in FIG. 2.

| Time | Control | % Remaining | ODS | % Remaining |
|---|---|---|---|---|
| mins | Tablet (μS/cm³) | (Control) | (μS/cm³) | (ODS) |
| 0.00 | 52.5 | 100% | 48.8 | 100% |
| 5.00 | 54.2 | 92% | 50 | 96% |
| 10.00 | 54.7 | 90% | 50.7 | 94% |
| 15.00 | 55.2 | 87% | 51.4 | 91% |
| 20.00 | 55.8 | 85% | 51.9 | 90% |
| 30.00 | 56.7 | 80% | 52.8 | 87% |
| 60.00 | 59.1 | 69% | 54.8 | 80% |
| 90.00 | 68.2 | 27% | 56.5 | 75% |
| 120.00 | 72 | 9% | 57.9 | 70% |
| 150.00 | 74 | 0% | 59.3 | 65% |
| 180.00 | 74 | 0% | 60.7 | 61% |
| 210.00 | 74 | 0% | 61.7 | 58% |
| 240.00 | 74 | 0% | 63 | 53% |
| 270.00 | NA | NA | 64.9 | 47% |
| 300.00 | NA | NA | 65.8 | 44% |
| 330.00 | NA | NA | 67.4 | 39% |
| 360.00 | NA | NA | 69.1 | 33% |
| 390.00 | NA | NA | 71 | 27% |
| 420.00 | NA | NA | 72.5 | 22% |
| 450.00 | NA | NA | 74.3 | 16% |
| 480.00 | NA | NA | 76.1 | 10% |
| 510.00 | NA | NA | 77.4 | 6% |
| 540.00 | NA | NA | 79.2 | 0% |
| 570.00 | NA | NA | 79.2 | 0% |
| 600.00 | NA | NA | 79.2 | 0% |

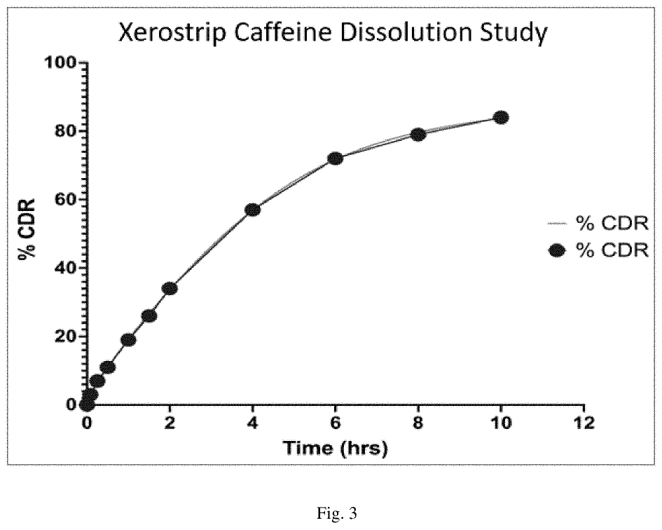

In vitro dispersion studies also confirm the steady release of active ingredients from the present ODS. One dispersion study determined that the rate of release was relatively constant over an 8-hour period (measured in % CDR or % cumulative drug release). To conduct this study, a small amount, roughly 30% of xylitol in the ODS was replaced with caffeine. Xylitol is difficult to study quantitatively when using high-performance liquid chromatography, HPLC, primarily because xylitol will appear at the same absorbance to other chemicals in the mixture resulting in an overlapping absorbance and inaccurate determination of release. Caffeine is a great surrogate because its profile under an HPLC analysis does not get confused with other substances and can be tracked with greater accuracy. Caffeine was a surrogate added to simplify the HPLC studies. The release rate was also determined to be a 1st order decay function as can be seen from FIG. 3.

The data found in the graph making up FIG. 3 can be seen in the table below:

| Time (hr) | CDR (%) |
|---|---|
| .1 | 4% |
| .25 | 7% |
| .5 | 12% |
| 1 | 22% |
| 1.5 | 29% |
| 2 | 36% |
| 4 | 57% |
| 6 | 71% |
| 8 | 75% |
| 10 | 78% |

Along with dispensing xylitol there are other applications for the present ODS. Many diseases require continual release of active compound to treat certain illnesses. Some potential examples of continuous localized delivery of oral care medication classifications include: Analgesics/Pain management, antibiotics, antifungal, antimicrobials, cough suppressant, dental/oral care, emergency medications, Endocrine/Metabolism, Gastrointestinal, Antiemetics, Hematology/Oncology, Neurologic, Obstetrics/Gynecology, Psychiatric, Esophagitis etc. The present disclosure may also be used with high dose systemic drug delivery for patients with dysphasia.

Patients at different stages of treatment may also obtain a benefit by using the present disclosure. For example, high dose local or systemic delivery for unconscious patients especially during emergency situations, combat, or for home care may have important medications delivered to them orally via the present ODS. The present disclosure may also be used to treat wounds not necessarily infectious diseases. Wound care applications include, burn care applications, surgical implants, oral surgical treatments, treatment of other oral diseases or developed from chemotherapy example (for example thrush, oral mucositis, & periodontal disease).

Thrush is a fungal infection caused by the yeast Candida. Thrush can affect various parts of the body, but most commonly occurs in the mouth and genital area. In the mouth, thrush may appear as white, creamy patches on the tongue, inner cheeks, and roof of the mouth. Thrush patches can be painful and may bleed if scraped or brushed. In severe cases, the infection can spread to the throat and esophagus, which may cause difficulty swallowing.

A constant supply of fluconazole, itraconazole, or Posaconazole to an affected area may cure thrush. If the present disclosure is loaded with fluconazole, itraconazole, or Posaconazole as the active ingredient, the constant release of the aforementioned drugs can greatly decrease the patches caused by thrush.

Oral mucositis is a common condition associated with cancer patients and those undergoing chemotherapy or radiation. Mucositis is characterized as a painful inflammation of the mucosa. Mucosa is the protective mucous membrane that lines the entire gastrointestinal tract, including lining of the mouth and intestines. Mucous membranes line many cavities and canals in the body, but mucositis in particular affects the digestive system, especially the oral mucosa. Oral mucositis is a common side effect of certain cancer treatments, including chemotherapy, radiation therapy, stem cell transplants or bone marrow transplants. It is hypothesized that because many cancer treatments target cells that divide rapidly, and the cells of the mucosa divide rapidly, these treatments can harm cells of the mucosa.

The present ODS can help with the treatment of oral mucositis by being loaded with salivatory stimulant such as xylitol and lubricants such as glycerol. The prolonged release of these ingredients provides a better treatment than conventional methods, because conventional methods introduce such ingredients to the oral cavity for only a limited amount of time, whereas the present ODS can release stimulants and lubricants for 8 hours or more.

Along with the side effects of cancer such as dry mouth, cancer itself may be treated with the same technology by delivering of cancer drugs. For example, high dose oral chemotherapy drugs may be delivered via the present ODS placed in a patient's oral cavity. Alkylating agents used in chemotherapy drugs may be used with the present ODS. These drugs include Altretamine, Bendamustine, Busulfan, Carboplatin, Carmustine, Chlorambucil, Cisplatin, Cyclophosphamide, Dacarbazine, Ifosfamide, Lomustine, Mechlorethamine, Melphalan, Oxaliplatin, Temozolomide, Thiotepa, Trabectedin.

The ODS may also be applied to areas of the body besides the oral cavity. For example, many birth control and vaginal disease treatments involve distributing certain drugs to the vaginal canal. The ODS may be used as a vaginal implant, birth control, female genital treatment or used for daily care such as cleaning. Appropriate drugs that may be used with the present ODS in this context include miconazole, tioconazole, metronidazole, clindamycin, terconazole, clotrimazole, povidone iodine, butoconazole, hydroxyquinoline, and sulfanilamide to name a few. The present ODS only needs 300-400 mg of Xylitol to work whereas other products may require 500 mg or above.

In certain instances, the desired therapeutic effect can be attained using lower dosing because of better bioavailability. In pharmaceutical chemistry, a smaller dose of medication as opposed to a larger dose to achieve the same concentration is preferred. This is due to a number of reasons. For one safety, a smaller dose reduces the risk of adverse effects. Many drugs may have harmful side effects, these side effects may be reduced or entirely eliminated if taken in smaller amounts. Using a smaller dosage may also be economically advantageous especially for expensive drug.

The present disclosure relates to an oral delivery system (ODS or oral dispersing strip). Other oral delivery systems use other technology methods for delivering drugs, nutrients, active ingredients, liquids, or other substances through the mouth to be absorbed into the body. One form is the tablet or capsule. Traditional tablets and capsules are very common oral delivery systems and involve the drug or active ingredient being enclosed in a solid form that is swallowed whole. Liquids are another Oral Delivery System and may be in the form of suspensions, solutions, and syrups. Liquids are often used for drugs that are not easily absorbed in solid form but must be absorbed relatively quickly (in under an hour). Powders are often used for drug delivery for drugs that are not stable in liquid form. They can be mixed with a liquid or food and swallowed, or sometimes combined with other oral delivery systems. However, in recent years there has been a growing use of chewable oral delivery products by both children and adults. Examples include lozenges and dissolvable tablets that are designed to dissolve in the mouth and deliver a drug as saliva is being swallowed. These Oral Delivery Systems are not suitable for sleep time use. It should be noted that present ODS, ODS, XODS, or reference to present disclosure or present ODS in this disclosure may refer to the specific disclosure of this application rather than other oral delivery systems as understood in the art (for example the tablets, liquids, and chewables discussed above).

Two common ways of oral drug delivery is through drug absorption within the oral cavity itself via transmucosal pathways or by swallowing the drug whole and following the more conventional gastrointestinal (GI) paths involving mucosa in the stomach and intestinal tract.

The rate and extent of oral drug absorption depends on several factors, including the physicochemical properties of the drug, the independent properties of the mucosa, and the presence of other chemicals and structures present at the absorption site. An advantage of oral absorption is that it avoids the effects of first-pass metabolism whereas GI absorption does not. Avoiding first-pass metabolism may mean dosing with less drug while attaining the same bioavailability level.

There are several types of membranes in the oral cavity that drugs can interact with during absorption. For one the buccal membrane is the membrane on the inside of the cheek. Drugs can be absorbed through the buccal membrane if designed to be placed between the cheek and gum. The sublingual membrane is another entry point for drugs administered orally. The sublingual membrane is the membrane under the tongue. Drugs can be absorbed through this membrane if designed to be placed under the tongue and left to dissolve. The palatal membrane is the membrane on the roof of the mouth. Different drugs may have different affinities for being absorbed via different membranes. Additionally, other drugs may be able to be absorbed via all the membranes. It should be noted that in this disclosure the word drug and active ingredient and active may be used interchangeably.

A matrix may be considered a material or system that contains multiple components or phases that are closely interconnected or intermixed. These components or phases can be solids, or liquids, and may have different physical or chemical properties. The matrix making up the present ODS may at certain times be considered a liquid (as binding factors and active ingredient are being added to it) a semi-solid (when the film matrix is drying or being heated in an oven) or a solid (the final present ODS).

Emulsifiers, also called an emulgent, are chemicals that assist in allowing two or more immiscible liquids (for example oil and water) to mix together to form a stable emulsion. Emulsifiers operate by reducing the surface tension between the two liquids, allowing the two liquids to form a stable mixture. Without an emulsifier, the two or more liquids would separate over time. Emulsifiers come in many different forms and are sometimes considered natural or synthetic. Emulsifiers are used in a wide range of products (but not for the purpose of binding other chemicals or causing a stable secretion of active ingredient), including foods, cosmetics, and pharmaceuticals. Some common natural emulsifiers include lecithin, which is found in egg yolks and soybeans, and gum arabic, which is derived from the sap of acacia trees. Synthetic emulsifiers, such as polysorbate 80 and sodium lauryl sulfate, are also widely used in industry.

A problem with the above emulsifiers is that they themselves may react with an active ingredient or drug found in a capsule. Worse yet, many emulsifiers may cause digestion problems or react with saliva in the mouth. Many use emulsifiers not for their properties of allowing liquids to diffuse slowly but for other properties. For example, emulsifiers may be used to create smooth and consistent textures in products such as mayonnaise, salad dressings, and ice cream. In cosmetics, emulsifiers may help to mix oil and water-based ingredients to create creams and lotions.

Glycerin, (also called glycerol), may come in a form as a colorless, odorless, sweet-tasting liquid. Glycerin has many applications but is not traditionally considered or used as an emulsifier. Glycerin is technically an alcohol with three hydroxyl (—OH) groups that account for its versatility as a compound. Glycerin is commonly produced by the hydrolysis of fats and oils, such as vegetable oil or animal fat. It is also used as a solvent, a humectant (a substance that helps to attract and retain moisture), and a sweetener. Glycerin may serve as a base emulsifier or a base substance from which the rest of the binders and chemicals are added to form the final ODS product. Other appropriate artificial sweeteners include aspartame, saccharin, sucralose acesulfame potassium, neotame, Advantame, cyclamate, and alitame. Other non-artificial sweeteners include honey maple syrup agave nectar coconut sugar, date sugar, molasses, fruit juices, and brown rice syrup.

Glycerin to date has not been considered an emulsifier. However, when mixed with the other chemicals in the present disclosure, it has been found to act as an emulsifier. It is hypothesized that because glycerin is a humectant when combined with the other chemicals discussed below, its ability to attract and retain moisturizer stabilizes the surface tension of other chemicals, thus allowing glycerin to act as an emulsifier. Glycerin can stabilize emulsions by forming hydrogen bonds with water molecules, which keep the oil, lipids, or non-water phases of a solution and water phases of the emulsion from separating. Glycerin itself may be considered a sweetener.

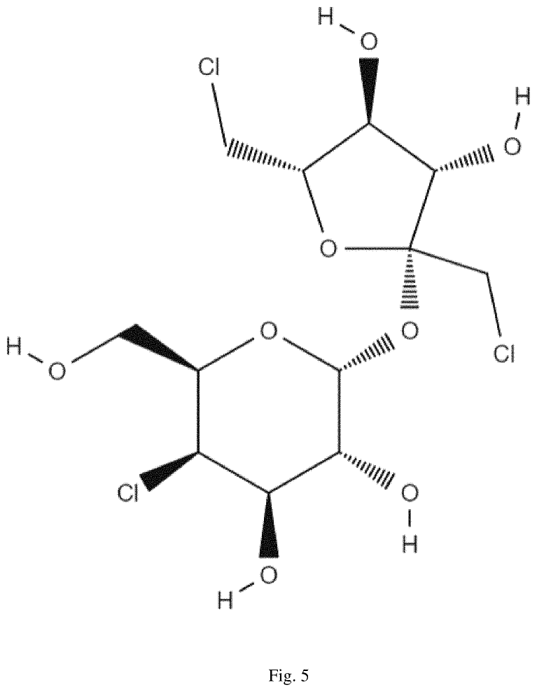

Sucralose is an artificial sweetener and sugar substitute that may be used as a sugar alternative in a variety of food and beverage products. A picture of the chemical structure of sucralose may be seen in FIG. 5, note the position of the chlorine atoms which will be discussed later on. It is derived from sugar and may be hundreds of times sweeter than regular sugar. Sucralose's molecular formula is C₁₂H₁₉Cl₃O₈. Sucralose's molecular structure may be thought of as a molecule of sucrose that has been modified by the addition of three chlorine atoms. Specifically, the hydroxyl groups (—OH) on the 1^{st}, 4^{th}, and 6^{th}, carbon atom of the sucrose molecule have been replaced by chlorine atoms (—Cl). The replacement of these hydroxyl groups with chlorine atoms makes sucralose much more stable than sucrose and contributes to sucralose' s sweetness. The presence of the chlorine atoms results in a more difficult molecule for the body to absorb, which contributes to it being a non-caloric food additive.

Sucralose contributes to the stability of the matrix and slow release of active ingredient for a number of reasons. First, sucralose is excellent for masking the unpleasant taste of particular drugs, making them more palatable for patients, especially when slow released to the mouth. Because sucralose is so much sweeter than regular sugar, less sucralose needs to be added to the solution (than if regular sugar was added) allowing for a smaller ODS. A smaller ODS is more comfortable for pediatric and geriatric populations who may have difficulty swallowing pills or tolerating bitter or unpleasant tasting drugs.

Second sucralose contributes to the sustained release of active ingredients because sucralose is a highly water-soluble compound that can form a stable matrix when combined with other excipients. The matrix controls the release rate of drugs or active ingredient over time, by controlling the diffusion of the drug molecules out of the matrix. Sucralose helps to maintain constant and slow diffusion of the drug molecule for longer periods of time, by contributing to the overall structure of the matrix. Unlike other molecules that may be found within the matrix and dissolve as time goes by, sucralose molecules are difficult to breakdown. As other polymers and chemicals (discussed below) breakdown as they are dissolved in the mouth, sucralose may hold the chemicals together (the chlorine in its structure are relatively inert and prevent it from breaking its bonds with other chemicals) while allowing sucrose to hold up the breaking down matrix.

A viscoelastic polymer may be Hypromellose otherwise known as hydroxypropyl methylcellulose (HPMC). Hypromellose is a semisynthetic, inert, viscoelastic polymer that has been used with eye drops. It may have a chemical formula of CH₂CH(OH)CH₃. Hypromellose may also be referred to as Hydroxypropyl methylcellulose, hydroxypropyl methyl cellulose HPMC; E464.

Viscoelastic polymers may be solid and have a white to beige coloring. The solid may be shaped into granules. When dissolved in liquid or water, hypromellose and other viscoelastic polymers and others will form colloids in solution. Hypromellose and other viscoelastic polymers used should be non-toxic. It should be noted that hypromellose can combust and may react strongly when introduced to other oxidizing agents directly. This oxidation is why it is important to mix hypromellose as instructed by this disclosure.

Colloids are a type of mixture where particles of one ingredient are dispersed throughout another ingredient. Unlike other ingredients or chemicals in a solution, colloidal particles are generally larger and do not dissolve completely in a surrounding solution. Instead, colloids remain suspended and dispersed evenly throughout the liquid. Colloids can exhibit unique optical, electrical, and mechanical properties, and in the experience of the inventors contribute to why active ingredient may be released at a slower rate in the present disclosure compared to other oral dissolvable delivery systems. It should be noted that although technically a colloid is not part of a solution, for purposes of this disclosure and for ease of reference if a colloid is in solution and reference is made to the solution, the colloid is included in that reference. Thus, a colloid may be considered part of the solution (because it is in solution) rather than actually being chemically part of the solution.

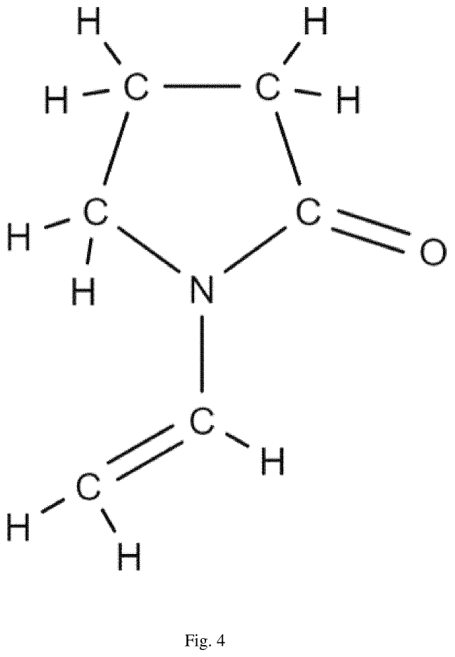

Vinylpyrrolidone (also called N-Vinylpyrrolidone) is an organic compound. FIG. 4 shows a diagram of the chemical structure of a molecule of vinylpyrrolidone with hydrogen atoms. Vinylpyrrolidone's chemical structure is that of a five membered cyclic amide derived from an amino alkanoic acid (also referred to as a lactam). Naturally, vinylpyrrolidone may appear as a colorless liquid, but in industry it may appear yellowish due to added coloring. Vinylpyrrolidone may undergo a polymerization reaction to yield polyvinylpyrrolidone (also known as PVP).

Polyvinylpyrrolidone (PVP), is also referred to as polyvidone or povidone. It is a water soluble polymer created from the polymerization of monomer N-vinylpyrrolidone. Depending on how many polymers or side chains are present in the PVP as well as what other constituents are mixed with it in a matrix, PVP may have a range of molecular weights and viscosities. The addition or subtraction of PVP can either increase or decrease the viscosity of the final matrix.

For purposes of the present disclosure polyvinylpyrrolidone is a water-soluble polymer derived from vinylpyrrolidone. Using a water soluble polymer is favorable because water soluble substances can easily be absorbed and transported within the body. This helps with active ingredient uptake and retention.

Cellulose is an organic compound with a chemical formula of (C₆H₁₀O₅)n. It is also considered a polysaccharide. Physically, cellulose may consist of hundreds to thousands of β(1→4) linked D-glucose units bound to each other. The difference in branching patterns attributes to the different properties a cellulose molecule (and its derivatives) may have. Cellulose is a base molecule from which many other derivative molecules may be obtained.

One cellulose derivative is carboxymethylcellulose. Like cellulose, carboxymethylcellulose is a polysaccharide with branched rings of sugar. However, in Carboxymethylcellulose, cellulose makes up the backbone of the molecule while carboxymethyl groups (CH₂—COOH) are bound to some of the hydroxyl groups of the glucopyranose monomers found on the cellulose. Carboxymethylcellulose may come as a sodium salt.

Other appropriate cellulose derivatives that may be used in the present ODS include, methylcellulose, hydroxypropyl methylcellulose, sodium carboxymethylcellulose, microcrystalline cellulose, ethylcellulose, croscarmellose sodium, sodium starch glycolate, calcium carboxymethylcellulose, carboxymethylcellulose sodium, sodium alginate, polysorbate 80, hydroxyethylcellulose, sodium carboxymethyl starch, hypromellose, carboxymethylated starch, cellulose acetate, hydroxypropyl cellulose, cross-linked sodium carboxymethyl cellulose, sodium hyaluronate, maltodextrin.

A third binder may be composed of two different organic molecules, one of which is an acetate. For example, the third binder may be composed of 1-vinyl-2-pyrrolidone and vinyl acetate mixed together before being added to the slurry. Vinyl acetate is an organic compound with the formula CH₃CO₂CH═CH₂. Vinyl acetate is a useful molecule to develop the present ODS from because of unique characteristics regarding how its polymerization can be controlled. When using Vinyl acetate to engage in polymerization-induced self-assembly, other copolymers may be blocked via self-assembly with a selective solvent. During polymerization the first block of polymer is thought to be solvophilic while the second block is solvophobic. This causes different parts of the polymer chain to polymerize with different chemicals found in the matrix. If all the chemicals in the matrix randomly polymerized with each other, the structure would likely yield a result that did not diffuse active ingredient at a rate of more than 8 hours.

1-vinyl-2-pyrrolidone is also an organic molecule colorless liquid with a sweet, chloroform-like odor. 1-vinyl-2-pyrrolidone is insoluble in water; soluble in acetone, ethanol and many organic solvents and very soluble in diethyl ether. When combined with vinyl acetate, the viscosity of the third binder is increased. Poly(l-vinylpyrrolidone-co-vinyl acetate) 64 or PVP VA64 are also appropriate to use as a third binder. The third binder polymer may also be an appropriate copolymer. Other appropriate copolymers include Ethylene-vinyl acetate (EVA) copolymers, Methacrylic acid-methyl methacrylate, Polyvinylpyrrolidone, Hydroxypropyl methylcellulose, Polyethylene glycol, and other Poloxamers.

When hypromellose is in an aqueous solution, it displays a thermal gelation property similar to methylcellulose. This means that as the solution heats up to a critical temperature, it congeals into a semi-flexible mass that cannot flow. The critical temperature is typically lower when the HPMC solution concentration and the methoxy group concentration within the HPMC molecules are higher. The viscosity or flexibility of the resulting mass is directly proportional to the concentration of the methoxy group—the higher the concentration, the more viscous or less flexible the resulting mass becomes.

Along with the physical ODS, a method of making the ODS is hereby disclosed. One appropriate method of making the presently disclosed ODS is combining a base emulsifier with at least one flavor and sweetener to form an emulsifier-polymer complex. Once this complex has been formed adding 200 proof ethanol to the emulsifier-polymer complex forms an emulsifier-polymer-ethanol solution.

200 proof ethanol (also known as 100% pure ethanol) is a strong solvent that may disrupt the structure of an emulsifier when mixed with other substances. Adding 200 proof ethanol to an emulsion, can dissolve and extract the emulsifier from the interface, which may result in destabilization of the emulsion. The addition of the ethanol may cause a separation in the two phases, resulting in the formation of an oily layer on top of an aqueous layer. It may seem counter intuitive to add ethanol at this point, however by separating out different layers, the chemicals that are added later may react with different chemicals in a different way. Rather than having all the chemicals in solution react with each other, having different chemicals reacting in different layers results in a more stably formed matrix.

Once the 200 proof ethanol has been added, the glycerin-polymer-ethanol solution may be homogenized via an immersion blender to create a slurry. The slurry should be a thick, viscous mixture of solid particles suspended within liquid. The solid particles may vary in size and shape, and the liquid will be the ethanol. The concentration of solid may vary depending on how much polymerization was underwent with the preceding chemicals. The viscosity and stability of the slurry may be adjusted by changing the concentration and composition of the solid and liquid components. Other factors may affect the slurry. For one the density of the slurry may be determined by the concentration and properties of the solid particles and polymers within it as well as the type and amount of solvent used.

After homogenization adding and blending a viscoelastic polymer as a first binder to the slurry via an immersion blender may be performed. An immersion blender operates by utilizing a motor to rotate a blade at high speeds. The blade is attached to the end of the handheld blender, and typically has a sharp edge. When the blender is turned on and the blade is immersed in the matrix and other constituents to be blended, the blade may chop and blend the homogenized glycerin-polymer-ethanol solution, creating a smooth and consistent mixture.

Adding and blending a water-soluble polymer derived from vinylpyrrolidone as a second binder to the slurry via an immersion blender, occurs after adding the first binder. Adding and blending a third binder composed of a copolymer with at least two different organic molecules one of which is an acetate to the slurry via an immersion blender, may occur after adding the second binder. Adding and blending a cellulose derivative as a fourth binder to the slurry via an immersion blender to create a slurry-binder solution may occur after adding the third binder. Adding an active ingredient to the slurry-binder solution to create a slurry-binder complex solution occurs after adding the fourth binder.

In chemistry, a substrate is generally referred to as a molecule or compound that may be acted upon by an enzyme in a chemical reaction. An enzyme may bind to a substrate at a specific location referred to as the active site. At the active site, the substrate may then be catalyzed or converted into one or more products. The substrate is usually a molecule undergoing a chemical reaction in the presence of an enzyme, and the type of reaction that occurs depends on the specific enzyme and substrate involved. This is not the type of substrate as referred to in the present disclosure.

In terms of the present disclosure the substrates are a material or surfaces in which the polymer complex may be dried or cut into strips. For example, in other contexts a substrate may be a silicon wafer that serves as a base for the deposit of a film of metal or oxide. The choice of substrate has a significant impact on the properties of the deposited film layer, including the film's crystal structure, electrical conductivity, or adhesion strength.

To deposit the film, the substrate surface must be properly prepared to provide a clean and well-defined surface for the deposition process. This involves cleaning the surface of the substrate with solvents or reactive gases. Treating the surface with a plasma to create functional groups that promote adhesion of the deposited material may also be appropriate. The substrate may also be heated before the deposit of substrate to make a film in order to improve the quality and uniformity of the deposited layer.

Once the substrate has been properly prepared the step of providing a substrate surface to spread the slurry-binder complex solution over, and spreading the solution over the substrate to create an evenly spread film of slurry-binder film should be completed. The substrate may be an inert surface in which a thin film is deposited during the production of ODS strips.

There are multiple ways to spread the film over the substrate. Spin coating may be accomplished by placing the substrate on a spinning platform. A small stream of slurry-binder complex solution is dispensed onto the center of the substrate, while the substrate is spun at a high speed by the spinning platform. The centrifugal force generated by the spinning of the substrate spreads the slurry-binder complex solution outwards and forms a uniform thin film on the substrate. The doctor blade technique may also be utilized. For the doctor blade technique applying a blade or flat surface is used. The blade may be held perpendicular to the substrate and a small amount of the slurry-binder complex solution material is dispensed onto the substrate. The blade may then be dragged across the substrate and slurry-binder complex solution, spreading the material and creating a thin film. For the spray coating technique, the slurry-binder complex solution material is atomized into small droplets and sprayed onto the substrate using a spray gun or atomizer. The droplets of slurry-binder complex solution land on the substrate and form a thin film.

Dip coating is also appropriate and involves immersing the substrate in a chamber filled with the slurry-binder complex solution and then slowly lifting the substrate out. This allows the excess slurry-binder complex solution to drip off the substrate and create a thin uniform film layer.

Once the film has been formed over the substrate, the film should be placed in an oven at a temperature between 30-70° C. for at least five minutes. Putting the film in the oven causes the material to harden for two main reasons. First the heat from the oven causes the water and liquid in the matrix to evaporate. As the water evaporates, the matrix material loses its moisture content, and the remaining solid material of the matrix becomes harder. Second, the hardening process that occurs after heating is due to a chemical reaction that takes place between the water and other components in the matrix. When the polymers within the matrix lose the water molecules between them, the polymers may bind to each other creating an even harder matrix.

Trimming the harder matrix into individual strips may be accomplished via mechanical slicer or individual cutting. Different strips may be loaded with sample size or active ingredient of 100-500 mg of active ingredient. It should be noted that sample size and active ingredient may be used interchangeably. In other words, a sample size of 350 mg means 350 mg of active ingredient is present in the strip. The dimensions of the individual strip are important as a strip being too large may result in too high a dosage and patient discomfort, too small and not enough active ingredient will be present to have a desired effect. The dimensions 3.14 cm×1.07 cm to 5.05 cm×1.45 cm in the experience of the inventors is favorable. Furthermore, a thickness of the strip between 0.15 cm to 0.3 cm is also favorable.

Additionally, in certain variations of the present ODS the at least one strip is meant for a 100-500 mg sample size with the dimensions 3.14 cm×1.07 cm to 5.05 cm×1.45 cm and the thickness of the strip is between 0.15 cm to 0.3 cm. the 100-500 mg sample size means 100-500 mg of active ingredient. It should be noted that active ingredient or sample can also be non-pharmaceutical ingredients such as electrolytes, herbals such as honey, ginger, licorice root, apricot kernel, coltsfoot flower, ginseng root, aster root, mulberry root bark, marshmallow root, thyme, ivy leaf, aniseed/anise oil, slippery elm and elderberry and vitamins, minerals, sweeteners and flavorings.

Closing Comments

Throughout this description, the embodiments and examples shown should be considered as exemplars, rather than limitations on the apparatus and procedures disclosed or claimed. Although many of the examples presented herein involve specific combinations of method acts or system elements, it should be understood that those acts and those elements may be combined in other ways to accomplish the same objectives. With regard to flowcharts, additional and fewer steps may be taken, and the steps as shown may be combined or further refined to achieve the methods described herein. Acts, elements and features discussed only in connection with one embodiment are not intended to be excluded from a similar role in other embodiments.

As used herein, “plurality” means two or more. As used herein, a “set” of items may include one or more of such items. As used herein, whether in the written description or the claims, the terms “comprising”, “including”, “carrying”, “having”, “containing”, “involving”, and the like are to be understood to be open-ended, i.e., to mean including but not limited to. Only the transitional phrases “consisting of” and “consisting essentially of”, respectively, are closed or semi-closed transitional phrases with respect to claims. Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. As used herein, “and/or” means that the listed items are alternatives, but the alternatives also include any combination of the listed items.

1. An oral dispersing strip for delivering a drug to a patient in need thereof comprising: a matrix composed of a base emulsifier, at least one sweetener, a first polymer binder, a water-soluble polymer derived from vinylpyrrolidone as a second binder, a copolymer with at least two different molecules one of which is an acetate as a third binder, a cellulose derivative as a fourth binder, and an active ingredient.

2. The oral dispersing strip of claim 1 wherein the target drug is dispersed from the matrix over the course of at least 450 minutes.

3. The oral dispersing strip of claim 1 where the active ingredient is xylitol.

4. The oral dispersing strip of claim 1 wherein the base emulsifier is glycerin.

5. The oral dispersing strip of claim 1 wherein the first binder is composed of sucralose and dextrose and at least one other sweetener.

6. The oral dispersing strip of claim 1 wherein the second binder is composed of polyvinylpyrrolidone.

7. The oral dispersing strip of claim 1 wherein the third binder is 1-vinyl-2-pyrrolidone and vinyl acetate and the fourth binder is carboxymethylcellulose.

8. A method of generating an oral dispersing strip for delivering a drug over an extended period of time to a patient in need thereof via the oral cavity comprising:
combining a base emulsifier with at least one flavor and sweetener to form a emulsifier-polymer complex;
adding 200 proof ethanol to the emulsifier-polymer complex to form a emulsifier-polymer-ethanol solution;
homogenizing the glycerin-polymer-ethanol solution via an immersion blender to create a slurry;
adding and blending a viscoelastic polymer as a first binder to the slurry via an immersion blender;
adding and blending a water-soluble polymer derived from vinylpyrrolidone as a second binder to the slurry via an immersion blender;
adding and blending a third binder composed of a copolymer with at least two different organic molecules one of which is an acetate to the slurry via an immersion blender;
adding and blending a cellulose derivative as a fourth binder to the slurry via an immersion blender to create a slurry-binder solution;
adding and blending a cellulose derivative as a fourth binder to the slurry via an immersion blender to create a slurry-binder solution;
adding an active ingredient to the slurry-binder solution to create a slurry-binder complex solution;
providing a substrate surface to spread the slurry-binder complex solution over, and spreading the solution over the substrate to create an evenly spread film of slurry-binder film;
placing the film in an oven at a temperature between 30-70° C. for at least five minutes;
verifying that the film has solidified and waiting for liquid to evaporate from the film if the film has not yet solidified;
trimming the film into at least one strip.

9. The method of claim 8 wherein the base emulsifier is glycerin.

10. The method of claim 8 wherein the first binder is hypromellose.

11. The method of claim 8 wherein the second binder is polyvinylpyrrolidone.

12. The method of claim 8 wherein the third binder is 1-vinyl-2-pyrrolidone and vinyl acetate.

13. The method of claim 8 wherein the fourth binder is carboxymethylcellulose.

14. The method of claim 8 wherein the active ingredient is xylitol.

15. The method of claim 2 wherein the at least one strip is meant for a 100-500 mg sample size with the dimensions 3.14 cm×1.07 cm to 5.05 cm×1.45 cm and the thickness of the strip is between 0.15 cm to 0.3 cm.

16. An oral dispersing strip for delivering a drug to a patient in need thereof via an oral cavity comprising:
a matrix with the dimensions of 3.14 cm−5.05 cm×1.07 cm−1.45 cm;
wherein the matrix is composed of a base emulsifier, at least one sweetener, a first polymer binder, a second binder of a water-soluble polymer derived from vinylpyrrolidone, a copolymer with at least two different molecules one of which is an acetate as a third binder, a cellulose derivative as a fourth binder, and an active ingredient.

17. The oral dispersing strip of claim 16 wherein the thickness of a single strip is between 0.15 cm to 0.3 cm.

18. The oral dispersing strip of claim 16 wherein the first binder is hypromellose.

19. The oral dispersing strip of claim 16 wherein the second binder is polyvinylpyrrolidone.

20. The oral dispersing strip of claim 16 where in the third binder is 1-vinyl-2-pyrrolidone and vinyl acetate and the fourth binder is carboxymethylcellulose.
